(19) United States
(12) Patent Application Publication
(10) Pub. No.: US 2026/0355059 A1
(43) Pub. Date: Nov. 12, 2026

(54) SYRINGE FOR GENERATING PARTICLE BASED FORMULATIONS

(71) Applicant: WEST PHARMACEUTICAL SERVICES, INC., Exton, PA (US)

(72) Inventors: James G. GUTHLEIN, Malvern, PA (US); Michael SULLIVAN, Birdsboro, PA (US); Rajiv KUMAR, Chester Springs, PA (US)

(21) Appl. No.: 18/997,080

(22) PCT Filed: Jul. 21, 2023
(86) PCT No.: PCT/US2023/028371

§ 371 Date: Jan. 19, 2025

Related U.S. Application Data
(60) Provisional application No. 63/391,409, filed on Jul. 22, 2022.

(51) Int. Cl. A61J 1/20

(52) U.S. Cl. CPC A61J 1/2096; A61J 1/2037; A61J 1/2055; A61J 1/2058

(57) ABSTRACT

A syringe and associated methods for generating a particle-based formulation is described. The syringe includes a barrel with a microfluidic mixer, a seal, and piston. A displacement of the piston in a distal direction is configured to draw a first fluid from a first fluid source into a first chamber of the barrel and to draw a second fluid from a second fluid source into a second chamber of the barrel. A displacement of the piston in a proximal direction is configured to expel the first fluid and the second fluid respectively out of the first chamber and the second chamber and into the microfluidic mixer to mix the first and second fluid into the particle-based formulation.

### CROSS REFERENCE TO RELATED APPLICATIONS

This application claims the benefit of U.S. Provisional Patent App. No. 63/391,409, filed Jul. 22, 2022, the disclosure of which is hereby incorporated by reference herein.

### TECHNICAL FIELD

This application generally relates to generating particle-based formulations, particularly to a syringe for generating particle-based formulations.

### BACKGROUND

Generation of particle-based formulations, such as for biomedical applications, can be costly and can involve complicated, time consuming steps. For example, low temperature storage is necessary to preserve the stability of some nano-or micro-particle-based formulations, which can complicate supply chains and increase costs associated with the particle-based formulations. Further, current techniques for generating particle-based formulations can involve the use of complicated equipment and a number of consumable mixing components that can occupy lab space. These problems can limit or prevent point of care/use generation of particle-based formulations.

Accordingly, there exists a need for systems and methods for generating particle-based formulations reproducibly with high precision in a consolidated process with fewer operational steps and at lower costs. Further, there exists a need for systems and methods for generating particle-based formulations at point of care/use and at or near room temperature.

### SUMMARY

These needs are met, to a great extent, by a syringe for generating a particle-based formulation. The syringe includes a barrel defining a bore extending between a proximal end and a distal end of the barrel; a first inlet at the proximal end that is configured to be fluidly connected to a first fluid source and to the bore; a second inlet at the proximal end that is configured to be fluidly connected to a second fluid source and to the bore; and a microfluidic mixer fluidly connected to the bore and extending between the proximal end and the distal end. The syringe also includes a seal that fluidly seals the proximal end of the barrel. The syringe also includes a piston within the bore. A displacement of the piston in a distal direction is configured to draw a first fluid from the first fluid source into a first chamber of the syringe via the first inlet and is configured to draw a second fluid from the second fluid source into a second chamber of the syringe. A displacement of the piston in a proximal direction is configured to expel the first fluid and the second fluid respectively out of the first chamber and the second chamber and into the microfluidic mixer to mix the first and second fluid to generate particle-based formulation.

Implementations may include one or more of the following features. The microfluidic mixer may include a first inlet fluidly connected to the first chamber, where the first inlet is configured to receive the first fluid from the first chamber. The microfluidic mixer may include a second inlet fluidly connected to the second chamber, where the second inlet is configured to receive the second fluid from the second chamber. The microfluidic mixer may include a microfluidic channel and an outlet fluidly connected to the first inlet of the microfluidic mixer and to the second inlet of the microfluidic mixer via the microfluidic channel. The syringe may include a tip cap that is configured to seal an outlet of the barrel. The piston may segment the bore into a third chamber distal to the piston, and the outlet of the microfluidic mixer is configured to be fluidly connected to the third chamber. The outlet may include a one-way valve that allows the particle-based formulation to exit the outlet of the microfluidic mixer and that prevents or restricts the particle-based formulation from re-entering the outlet of the microfluidic mixer from the third chamber. The displacement of the piston in the proximal direction is configured to expel the particle-based formulation out of the outlet of the microfluidic mixer and into the third chamber. The displacement of the piston in the distal direction is a first displacement of the piston in the distal direction, and a second displacement of the piston in the distal direction subsequent to the first displacement of the piston in the distal direction and subsequent to the displacement of the piston in the proximal direction is configured to expel the particle-based formulation via an outlet of the barrel. The plunger rod, the piston, and the seal segment the bore proximal to the piston into the first chamber and the first chamber is fluidly connected to the first inlet and to the microfluidic mixer. The plunger rod, the piston, and the seal segment the bore proximal to the piston into the second chamber and the second chamber is fluidly connected to the second inlet and to the microfluidic mixer. The plunger rod may include a gasket that prevents fluid communication between the first chamber and the second chamber. The plunger rod extends through an interface of the seal, where the interface has a complimentary shape to a shape of the plunger rod to prevent rotation of plunger rod within the barrel. The plunger rod is removably attached to the piston. The first chamber has a first volume and the second chamber has a second volume, where the first volume and the second volume vary in response to displacement of the piston within the bore. The first inlet and the second inlet define respective Luer locks. The syringe may include a first plug that is configured to plug the first inlet and a second plug that is configured to plug the second inlet. The first fluid may include lyophilized mRNA and the second fluid may include a lyophilized lipid. The syringe may include a needle configured to be attached to an outlet of the barrel. The seal defines an end cap.

Another aspect includes a method of generating a particle-based formulation in a syringe. The method of generating includes connecting a first fluid source to a first inlet of the syringe and a second fluid source to a second inlet of the syringe. The generating also includes displacing a piston of the syringe in a distal direction to draw a first fluid from the first fluid source into a first chamber of a barrel of the syringe via the first inlet and to draw a second fluid from the second fluid source into a second chamber of the barrel via the second inlet. The first and second chambers each being located proximal to the piston. The generating also includes displacing the piston in a proximal direction to expel the first fluid from the first chamber and into a microfluidic mixer of the syringe and to expel the second fluid from the second chamber and into the microfluidic mixer of the syringe thereby mixing the first fluid and the second fluid in the microfluidic mixer to generate the particle-based formulation.

Implementations of this other aspect may include one or more of the following features. The displacing of the piston in the proximal direction expels the particle-based formulation out of the microfluidic mixer and into a third chamber of the barrel that is located distal to the piston. Displacing the piston in the distal direction a second time to expel the particle-based formulation out of an outlet of the barrel. Attaching a needle to the outlet of the barrel prior to displacing the piston in the distal direction the second time. The method may include removing, subsequent to displacing the piston in the proximal direction, a tip cap from an outlet of the barrel. The method may include prior to the displacing the piston in the proximal direction, removing the first fluid source from the first inlet and the second fluid source from the second inlet; and connecting a first plug to the first inlet and a second plug to the second inlet.

The first fluid may include lyophilized mRNA and the second fluid may include a lyophilized lipid.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a perspective view of a syringe;

FIG. 2 shows the syringe of FIG. 1 connected with a first and a second fluid source and with a plunger rod arranged in a proximal position;

FIG. 3 shows the syringe of FIG. 1 with the plunger rod moved into a distal position and with the first and second fluid source respectively replaced with first and second plugs;

FIG. 4 shows the syringe of FIG. 1 with the plunger rod again moved into the proximal position to mix a first and second fluid into a particle-based formulation within a microfluidic mixer of the syringe;

FIG. 5 shows the syringe of FIG. 1 with the plunger rod again moved into the distal position to expel the particle-based formulation from the syringe; and

FIG. 6 shows a method of generating a particle-based formula.

### DETAILED DESCRIPTION OF ILLUSTRATIVE EMBODIMENTS

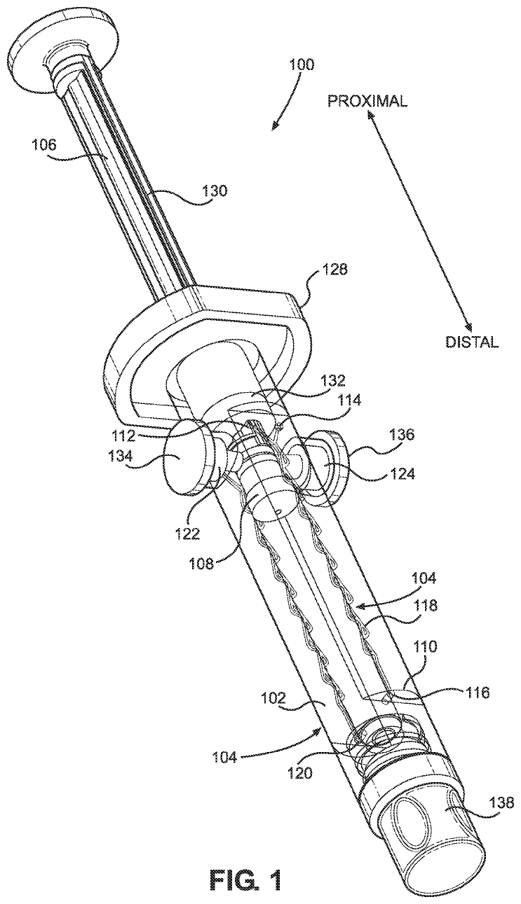

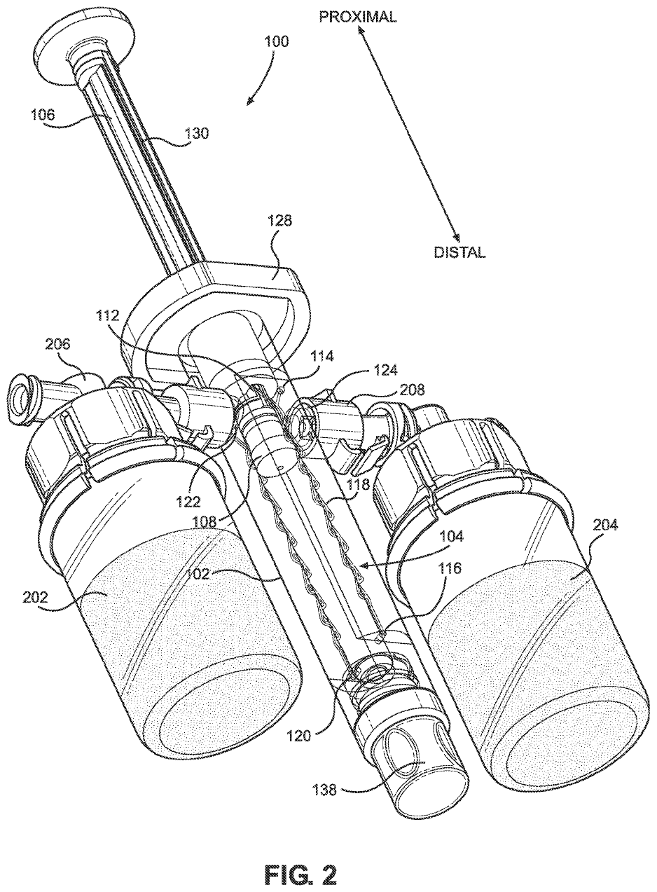

FIG. 1 shows a syringe 100 for generating a particle-based formulation. FIGS. 2-5 show an example use of the syringe 100. The syringe 100 can include a barrel 102 that can define a microfluidic mixer 104, such as a microfluidic chip. The syringe 100 can further include a plunger rod 106 with a piston 108 attached to a distal end of the plunger rod 106. Although a plunger rod 106 is described, there can be other ways to move the piston 108 within the barrel 102 and accordingly the syringe 100 can be provided without a plunger rod 102. In embodiments, the plunger rod 106 can be removably attached to the piston 108. As discussed in greater later with respect to FIGS. 2-5, displacement of the plunger rod 106 and the piston 108 in a distal direction to a distal position can simultaneously draw a first fluid and a second fluid into the syringe 100. Subsequent displacement of the plunger rod 106 and the piston 108 in a proximal direction from the distal position to a proximal position can expel the first and second fluid into the microfluidic mixer 104 to mix the first and second fluid into the particle-based formulation. The term “liquid” as used herein can refer to any free flowing substances and can include solid substances suspended in free flowing solutions. The term “particle-based formulation” can include formulations of two or more substances and can include nanoparticles and/or microparticles. Subsequent displacement of the plunger rod 106 and the piston 108 in the distal direction from the proximal position to the distal position can expel the particle-based formulation from the syringe 100.

According to aspects of the invention the syringe 100 can allow particle-based formulations to be generated at point of use with minimal steps and without the need of cumbersome, extraneous equipment. In embodiments, each of the first and second fluids can be lyophilized components and the syringe 100 can mix the reconstituted lyophilized components stored at room temperature (e.g., a temperature between 10 °C and 30 °C) to generate a particle-based formulations, such as drugs, vaccines, among other possibilities. For example, the first and/or second fluids can include any combination of polymers, lipids, combination of lipids and polymers, small molecule drugs, proteins, nucleic acids, ASO, gene editing formulations such as CRISPR/cas9, mRNA, siRNA, saRNA, among other possibilities. In embodiments, the first fluid can include lyophilized mRNA, which can be reconstituted in a buffer. In embodiments, the second fluid can be a lyophilized lipid mixture, which can be reconstituted in an organic solvent such as ethanol. The syringe 100 can be used for a number of polymer-based nano particles and microparticles and for mRNA drugs or other materials containing small molecules. The syringe 100 can generate the particle-based formulation with high precision and high repeatability and can be used in biomedical applications to generate high potency drugs, vaccines, etc. By eliminating extraneous equipment and by allowing particle-based formulations to be generated at point of use and at room temperature, the syringe 100 can significantly reduce costs associated with the generation of particle-based formulations and increase the accessibility of the particle-based formulations.

Returning to FIG. 1 and as discussed previously, the syringe 100 can include the barrel 102, which can define a microfluidic mixer 104. In embodiments such as shown in FIGS. 1-5, the barrel 102 can be translucent to allow for visual inspection of the interior of the barrel 102. Additionally or alternatively, some or all of the barrel 102 can be opaque. In embodiments, the microfluidic mixer 104 can be integrally formed with the barrel 102. For example, the barrel 102 can include a planar or face region at an outer surface of the barrel 102 and the microfluidic mixer 104 can be etched into the planar region. The barrel 102 can include a cover 110 that can cover and seal the microfluidic mixer 104 from an external environment surrounding the syringe 100. In embodiments, the cover 110 can be removable. Alternatively, the cover 110 can be immovably fixed to the barrel 102. The microfluidic mixer 104 can define a first inlet 112, a second inlet 114, an outlet 116, and a microfluidic channel 118 fluidly connecting the outlet 116 with each of the first inlet 112 and the second inlet 114. In embodiments, the microfluidic mixer 104 can include one or more valves that can regulate fluid flow through the microfluidic mixer 104. For example, the outlet 116 can include a one-way valve that can allow particle-based formulation to exit the outlet 116 and that can prevent the particle-based formulation from re-entering the outlet 116. Additionally or alternatively, the first inlet 112 and/or the second inlet 114 can include one-way valves that can prevent fluid from exiting out of microfluidic mixer 104 via the first inlet 112 and/or the second inlet 114. One-way valves as described in this disclosure can be passive, such as duck-bill valves, or active, such as a stopcock valve.

In embodiments, the syringe 100 can include two or more microfluidic mixers 104. The microfluidic mixers 104 can have the same features and can interface other structures of the syringe 100 as discussed throughout this disclosure. In alternative embodiments, the syringe 100 can have only one microfluidic mixer 104.

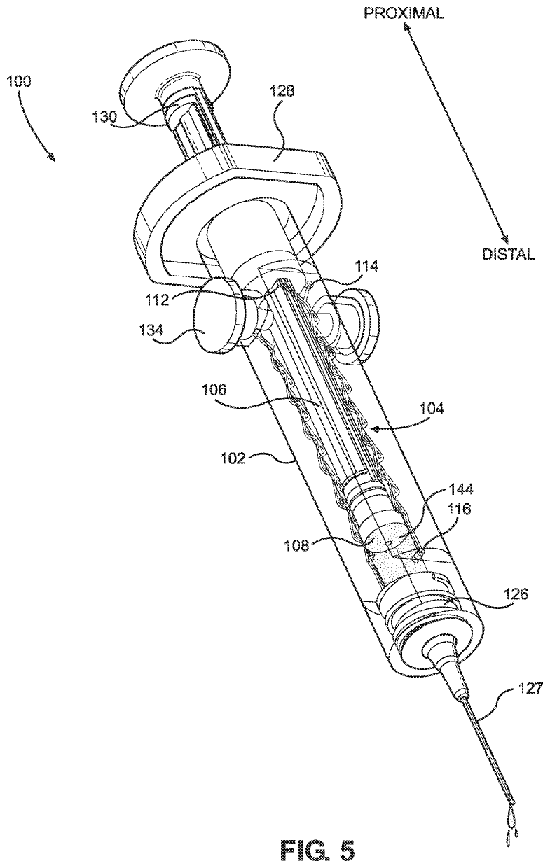

The barrel 102 can define a bore 120 extending between a proximal end and a distal end of the barrel 102. As shown for example in FIG. 1, the barrel 102 can define a first inlet 122 and a second inlet 124 that can each be located at the proximal end of the barrel 102. For example and as shown in FIG. 5, the barrel 102 can define an outlet 126 that can be located at the distal end of the barrel 102. The first inlet 122, second inlet 124, and outlet 126 can be fluidly connected to the bore 120. In embodiments, any or all of the first inlet 122, second inlet 124, and outlet 126 can include a fitting, such as for example a Lure lock, that can connect any of the first inlet 122, second inlet 124, and outlet 126 to one or more other structures. For example and as shown in FIG. 2, the first inlet 122 can be fluidly connected to a first fluid source 202 and the second inlet 124 can be connected to a second fluid source 204 via respective first and second connectors 206, 208. In embodiments, the first and second fluid sources 202, 204 can for example define vials for respectively containing the first and second fluids. In embodiments, the syringe 100 can include more than two inlets and can be fluidly connected to more than two fluid sources to mix more than two fluids together into a particle-based formulation.

In embodiments, the outlet 126 can be fluidly connected to a vial, needle, or other assembly. For example, the outlet 126 can be connected to a vial that collects the particle-based formulation for end use applications. Additionally or alternatively, in the embodiment of FIG. 5 the outlet 126 can be fluidly connected to a hollow needle 127 that can inject the particle-based formulation for end use applications. In embodiments, the particle-based formulation formed by the syringe 100 can be purified. For example, the particle-based formulation can be purified after exiting the outlet 126 and before the particle-based formulation is transferred to a patient or end user.

Some or all of the syringe 100 can at times be sealed from the external environment. For example, the syringe 100 can include a seal, such as an end cap 128, at the proximal end of the barrel 102. The plunger rod 106 can extend through the end cap 128 and together with the end cap 128 can seal the proximal end of the barrel 102. In embodiments, the plunger rod 106 can include a gasket 130 and/or the end cap 128 can include a gasket 132 that seal the proximal end of the barrel 102. The gasket 130 can be provided on a shaft of the plunger rod 106. The syringe 100 can include a first plug 134 and a second plug 136 that can respectively seal the first inlet 122 and the second inlet 124 from the external environment. The syringe 100 can include a seal component 138, such as a tip cap for a Luer lock or needle guard/shield, that can seal the outlet 126 of the barrel 102. Any or all of the end cap 128, plunger rod 106, the piston 108, the first plug 134, the second plug 136, and the seal component 138 can be removably connected to the syringe 100 via any number fitting or connections including for example snap fittings, threads, Luer locks, etc.

The bore 120 of the barrel 102 can be segmented into any number of chambers. Fluid can be isolated within and selectively communicated between the chambers in response displacement of the plunger rod 106 and the piston 108. In embodiments such as those shown in FIGS. 1-5, the bore 120 proximal to the piston 108 can be segmented into a first chamber 140 and a second chamber 142 and the bore 120 distal to the piston 108 can be segmented into a third chamber 144. For example, the plunger rod 106, the piston 108, and the end cap 128 can segment the bore 120 proximal to the piston 108 into the first chamber 140 and the first chamber 140 can be fluidly connected to the first inlet 122 and to the microfluidic mixer 104. The plunger rod 106, the piston 108, and the end cap 128 can segment the bore 120 proximal to the piston 108 into the second chamber 142. The second chamber 142 can be fluidly connected to the second inlet 124 and to the microfluidic mixer 104. The piston 108 can segment the bore 120 into the third chamber 144 distal to piston 108. The plunger rod 106, the piston 108, and the end cap 128 can form fluid tight seals with an inner surface of the bore 120 such that fluid can be isolated within the first chamber 140, the second chamber 142, and/or the third chamber 144. In embodiments, the gaskets 130, 132 can form the fluid tight seals and can segment the bore 120 into the first chamber 140, the second chamber 142, and the third chamber 144. When connected, the first and second plugs 134, 136 can respectively seal fluid within the first and second chambers 140, 142. When connected, the seal component 138 can seal fluid within the third chamber 144. In embodiments, the bore 120 can be segmented into more than two chambers proximal to the piston 108.

The volume of the first chamber 140, the second chamber 142, and the third chamber 144 can vary as the piston 108 is displaced within the bore 120. For example, the volumes of the first chamber 140 and the second chamber 142 can increase and the volume of the third chamber 144 can decrease as the piston 108 is displaced in the distal direction.

Conversely, the volumes of the first chamber 140 and the second chamber 142 can decrease and the volume of the third chamber 144 can increase as the piston 108 is displaced in the proximal direction. Though the volumes the first chamber 140, the second chamber 142, and the third chamber 144 can vary, relative volumetric relationships between the first chamber 140, the second chamber 142, and the third chamber 144 can be maintained. For example, in embodiments the volume of the first chamber 140 and the volume of the second chamber 142 can remain equal relative to each other throughout the displacement of the piston 108. This can allow for a 1:1 mixing ratio of first and second fluids within the microfluidic mixer 104.

Alternatively, in embodiments the volume of the first chamber 140 and the volume of the second chamber 142 can remain different by a fixed amount throughout displacement of the piston 108. According to these embodiments, the syringe 100 can be tuned to produce a variety of different mixing ratios of first and second fluids within the microfluidic mixer 104 such as for example 3:1. The syringe 100 can be tuned to produce different ratios by, for example, modifying the cross-sectional area of the chambers and/or by increasing or decreasing an axial geometry of the plunger rod 106.

In embodiments, the end cap 128 can include an interface 146 that prevents rotation of the plunger rod 106 within the barrel 102 about a longitudinal axis of the plunger rod 106. For example, the interface 146 can have a complementary shape to the shape of the plunger rod 106 to rotationally interlock the plunger rod 106 relative to the barrel 102. According to this configuration, chambers of the bore 120 (e.g., the first chamber 140 and the second chamber 142) segmented by the plunger rod 106 can remain rotationally fixed, which can improve and maintain intended fluid communication between the chambers and the microfluidic mixer 104.

In embodiments, structures of the syringe 100, such as the barrel 102, can be injection molded. Additionally or alternatively, structures of the syringe 100, such as the microfluidic mixer 104, can be laser cut.

FIGS. 2-5 show a use of the syringe 100 in which the syringe 100 generates a particle-based formulation from a first and second fluid. As shown in FIG. 2, syringe 100 can be provided in a first arrangement in which the plunger rod 106 is disposed in a proximal position. In the first arrangement, the first inlet 122 can be fluidly connected to the first fluid source 202 via the first connector 206 and the second inlet 124 can be fluidly connected to the second fluid source 204 via the second connector 208.

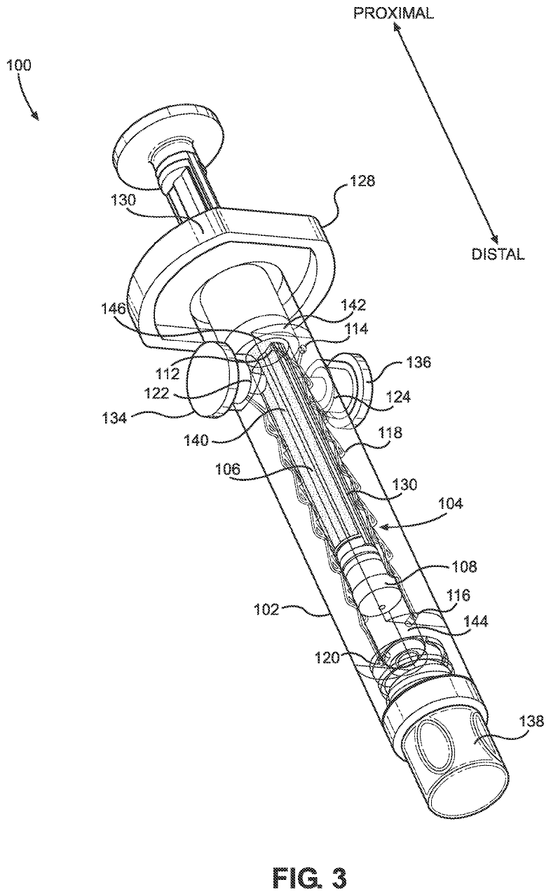

As shown in FIG. 3, the syringe 100 can be moved into a second arrangement after the first arrangement. Moving the syringe 100 into the second arrangement can involve a displacement of the plunger rod 106 and the piston 108 in a distal direction from the proximal position shown in FIG. 2 to a distal position shown in FIG. 3. This displacement in the distal direction can draw (i.e., fluidly communicate) a first fluid from the first fluid source 202 into the first chamber 140 via the first inlet 122 and can draw a second fluid from the second fluid source 204 into the second chamber 142 via the second inlet 124. As discussed above, the piston 108, the plunger rod 106, and the end cap 128 can together seal the first chamber 140 and the second chamber 142 and can prevent direct fluid communication between the first chamber 140 and the second chamber 142. Accordingly, the first fluid and the second fluid respectively contained within the first chamber 140 and the second chamber 142 do not mix in the second arrangement. In the second arrangement and after the first and second fluid are drawn into the syringe 100, the first connector 206 and the second connector 208 can be removed and the first plug 134 and the second plug 136 can be respectively connected to the first inlet 122 and the second inlet 124 to plug the first inlet 122 and the second inlet 124.

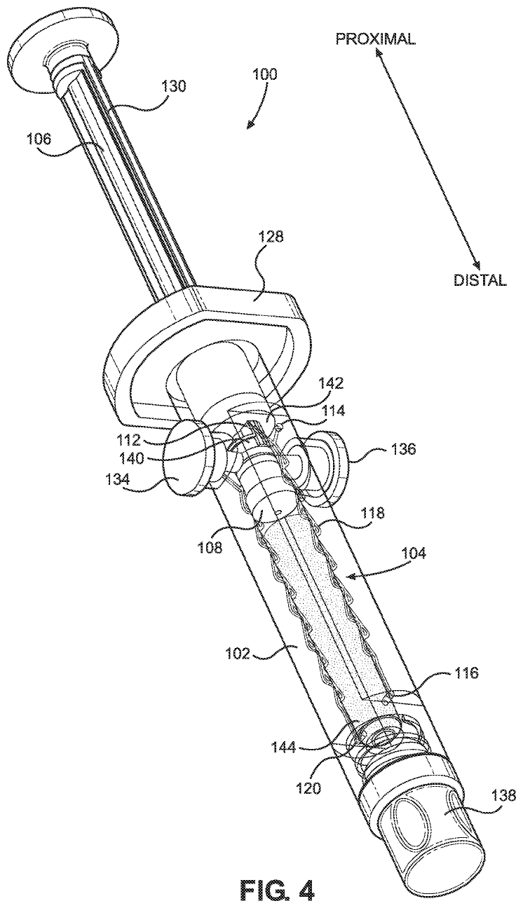

As shown in FIG. 4, the syringe 100 can be moved into a third arrangement after the second arrangement. Moving the syringe 100 into the third arrangement can involve a displacement of the plunger rod 106 and the piston 108 in the proximal direction from the distal position and back to a proximal position. This displacement in the proximal direction can cause the piston 108 to expel the first fluid and the second fluid respectively out of the first chamber 140 and the second chamber 142 and into the microfluidic mixer 104 to mix the first and second fluid into the particle-based formulation. For example, fluid communication between the microfluidic channels 118 and the first chamber 140 can be provided by the first inlet 112. Fluid communication between the microfluidic channels 118 and the second chamber 142 can be provided by the second inlet 114. Fluid communication between the microfluidic channels 118 and the third chamber 144 can be provided by the outlet 116. The first plug 134 and the second plug 136 can prevent fluid from exiting the first inlet 122 and the second inlet 124 when the piston 108 is displaced in the proximal direction. Additionally or alternatively, the first inlet 122 and/or the second inlet 124 can include one-way valves that can prevent fluid from exiting out of the first inlet 122 and/or the second inlet 124. This displacement of the piston 108 in the proximal direction can reduce the volume of the first chamber 140 and the second chamber 142 to respectively expel the first and second fluid from the first chamber 140 and the second chamber 142 respectively via the first inlet 112 and the second inlet 114. This displacement of the piston 108 in the proximal direction can also drive the first and second fluid through the first inlet 112 and the second inlet 114 and into the microfluidic channels 118 where the first and second fluid can mix together into a particle-based formulation within the geometry of the microfluidic channels 118. This displacement of the piston 108 can also expel the particle-based formulation from the microfluidic channels 118 and into the third chamber 144 via the outlet 116, which can fluidly connect the microfluidic mixer 104 and the third chamber 144.

As shown in FIG. 5, the syringe 100 can be moved into a fourth arrangement after the third arrangement. Moving the syringe 100 into the fourth arrangement can involve removing the seal component 138. In embodiments, removing a seal component 138 can reveal the hollow needle 127 for end use applications of the particles based formulations. Alternatively, after the seal component 138 is removed the outlet 126 can be connected to the hollow needle 127, a fluid line, or any other structure intended to receive and/or transfer the particle-based formulation. After removal of the seal component 138, moving the syringe 100 into the fourth arrangement can further involve another displacement of the plunger rod 106 and the piston 108 in the distal direction from the proximal position to a distal position. This displacement in the distal direction can expel the particle-based formulation out of the third chamber 144 via the outlet 126, which is fluidly connected to the third chamber 144. Once expelled from the syringe 100 the particle-based formulation can be used for any end use including injection into a patient, experimentation, storage, etc. In embodiments, the particle-based formulation formed by the syringe 100 can be purified. For example, the particle-based formulation can be purified after exiting the outlet 126 and before the particle-based formulation is transferred to a patient or end user.

In embodiments, displacement of the plunger rod 106 and the piston 108 can be performed manually and/or automatically. For example, the plunger rod 106 and the piston 108 can be displaced in response to a user manually pushing or pulling the plunger rod 106. In embodiments, a mechanical and/or electrotechnical actuator can be connected to the plunger rod 106 and displacement of the plunger rod 106 and the piston 108 can be controlled directly or indirectly (i.e., with a controller) by the actuator. The actuator can include any or all of a spring, rubber band, motor, gear train, etc.

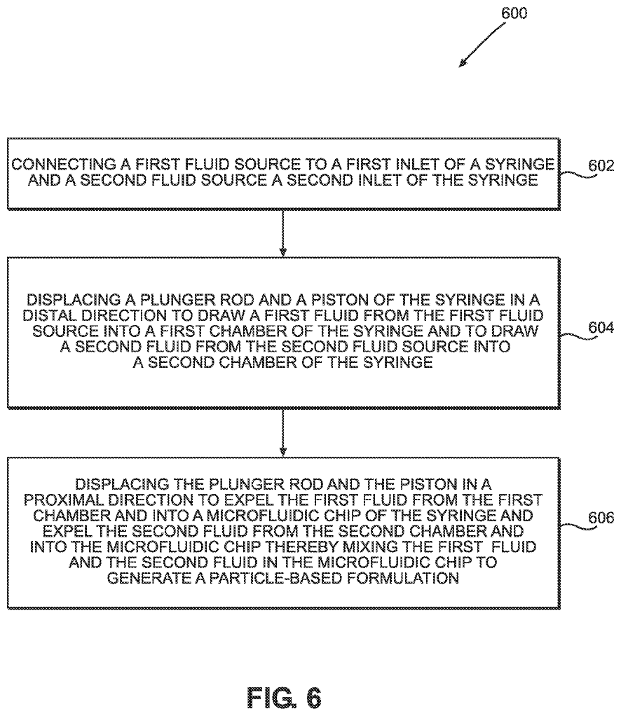

FIG. 6 shows a method 600 of generating a particle-based formulation using the syringe 100. The method 600 can include, at a step 602, connecting the first fluid source 202 to the first inlet 122 and the second fluid source 204 to the second inlet 124. Prior to connecting the first fluid source 202 to the first inlet 122 and the second fluid source 204 to the second inlet 124, the first fluid source 202 and the second fluid source 204 can be stored at a room temperature, such as a temperature between 10 °C and 30 °C. As discussed previously, a syringe 100 that can use first and second fluids stored at room temperature to generate particle-based formulations can simplify and reduce costs associated with the generation of particle-based formulations. The syringe 100 at step 602 can include any of the features/relationships discussed above with respect to the first arrangement and as depicted in FIG. 2.

The method 600 can include, at a step 604, displacing the plunger rod 106 and the piston 108 in a distal direction to draw the first fluid from the first fluid source 202 into the first chamber 140 via the first inlet 122 and to draw the second fluid from the second fluid source 204 into the second chamber 142 via the second inlet 124. As discussed above, in embodiments the first fluid can include lyophilized mRNA and the second fluid can include a lyophilized lipid. Step 604 can occur after step 602.

The method 600 can include, at a step 606, displacing the plunger rod 106 and the piston 108 in a proximal direction to expel the first fluid from the first chamber 140 and into a microfluidic mixer 104 and to expel the second fluid from the second chamber 142 and into the microfluidic mixer 104 thereby generating the particle-based formulation. Displacing of the plunger rod 106 and the piston 108 in the proximal direction can expel the particle-based formulation out of the microfluidic mixer 104 and into the third chamber 144. The syringe 100 after step 606 can include any of the relationships/features discussed previously in reference to the third arrangement and as depicted in FIG. 4.

The method 600 can also include removing, subsequent step 604 and prior to step 606, the seal component 138 from the outlet 126. The method 600 can also include displacing the plunger rod 106 and the piston 108 in the distal direction a second time to expel the particle-based formulation out of the third chamber 144 via the outlet 126. The method 600 can include, subsequent to displacing the plunger rod 106 and the piston 108 in the proximal direction, removing the seal component 138 (e.g., the tip cap) from the outlet 126 of the barrel 102. The method 600 can include, prior to displacing the plunger rod and the piston in the distal direction the second time, attaching the hollow needle 127 to the outlet 126 of the barrel 102.

After this second displacement, the syringe 100 can include any of the features/relationships discussed previously with respect to the fourth arrangement and as depicted in FIG. 5.

The method 600 can include, subsequent to step 604 and prior to step 606, removing the first fluid source 202 from the first inlet 122 and the second fluid source 204 from the second inlet 124 and connecting the first plug 134 to the first inlet 122 and a second plug 136 to the second inlet 124. The syringe in this position can include any of the features/relationships discussed previously in reference to the second arrangement and as depicted in FIG. 3.

It will be appreciated that the foregoing description provides examples of the disclosed machine. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated. All methods described herein can be performed in any suitable order unless otherwise indicated herein or otherwise clearly contradicted by context.

1. A syringe for generating a particle-based formulation, the syringe comprising:
a barrel defining:
a bore extending between a proximal end and a distal end of the barrel;
a first inlet at the proximal end that is configured to be fluidly connected to a first fluid source and to the bore;
a second inlet at the proximal end that is configured to be fluidly connected to a second fluid source and to the bore; and
a microfluidic mixer fluidly connected to the bore and extending between the proximal end and the distal end,
a seal that fluidly seals the proximal end of the barrel; and
a piston within the bore,
wherein a displacement of the piston in a distal direction is configured to draw a first fluid from the first fluid source into a first chamber of the syringe via the first inlet and is configured to draw a second fluid from the second fluid source into a second chamber of the syringe, and
wherein a displacement of the piston in a proximal direction is configured to expel the first fluid and the second fluid respectively out of the first chamber and the second chamber and into the microfluidic mixer to mix the first and second fluid to generate particle-based formulation.

2. The syringe of claim 1, wherein the microfluidic mixer comprises:
a first inlet fluidly connected to the first chamber, wherein the first inlet is configured to receive the first fluid from the first chamber;
a second inlet fluidly connected to the second chamber, wherein the second inlet is configured to receive the second fluid from the second chamber;
a microfluidic channel; and
an outlet fluidly connected to the first inlet of the microfluidic mixer and to the second inlet of the microfluidic mixer via the microfluidic channel.

3. (canceled)

4. The syringe of claim 2, wherein the piston segments the bore into a third chamber distal to the piston, and
wherein the outlet of the microfluidic mixer is configured to be fluidly connected to the third chamber.

5. The syringe of claim 4, wherein the outlet comprises a one-way valve that allows the particle-based formulation to exit the outlet of the microfluidic mixer and that prevents or restricts the particle-based formulation from re-entering the outlet of the microfluidic mixer from the third chamber.

6. The syringe of claim 4, wherein the displacement of the piston in the proximal direction is configured to expel the particle-based formulation out of the outlet of the microfluidic mixer and into the third chamber.

7. The syringe of claim 1, wherein the displacement of the piston in the distal direction is a first displacement of the piston in the distal direction, and
wherein a second displacement of the piston in the distal direction subsequent to the first displacement of the piston in the distal direction and subsequent to the displacement of the piston in the proximal direction is configured to expel the particle-based formulation via an outlet of the barrel.

8. The syringe of claim 1, further comprising a plunger rod attached to the piston, the plunger rod movably extending through the seal,
wherein the plunger rod, the piston, and the seal segment the bore proximal to the piston into the first chamber and the first chamber is fluidly connected to the first inlet and to the microfluidic mixer, and
wherein the plunger rod, the piston, and the seal segment the bore proximal to the piston into the second chamber and the second chamber is fluidly connected to the second inlet and to the microfluidic mixer.

9. The syringe of claim 8, wherein the plunger rod comprises a gasket that prevents fluid communication between the first chamber and the second chamber.

10. The syringe of claim 8, wherein the plunger rod extends through an interface of the seal, wherein the interface has a complimentary shape to a shape of the plunger rod to prevent rotation of plunger rod within the barrel.

11. (canceled)

12. The syringe of claim 1, wherein the first chamber has a first volume and the second chamber has a second volume,
wherein the first volume and the second volume vary in response to displacement of the piston within the bore.

13. The syringe of claim 1, wherein the first inlet and the second inlet define respective Luer locks.

14. The syringe according to claim 1, further comprising a first plug that is configured to plug the first inlet and a second plug that is configured to plug the second inlet.

15. The syringe according to claim 1, wherein the first fluid comprises lyophilized mRNA and the second fluid comprises a lyophilized lipid.

16. (canceled)

17. The syringe according to claim 1, wherein the seal defines an end cap.

18. A method of generating a particle-based formulation in a syringe, the method comprising:
connecting a first fluid source to a first inlet of the syringe and a second fluid source to a second inlet of the syringe;
displacing a piston of the syringe in a distal direction to draw a first fluid from the first fluid source into a first chamber of a barrel of the syringe via the first inlet and to draw a second fluid from the second fluid source into a second chamber of the barrel via the second inlet, the first and second chambers each being located proximal to the piston; and
displacing the piston in a proximal direction to expel the first fluid from the first chamber and into a microfluidic mixer of the syringe and to expel the second fluid from the second chamber and into the microfluidic mixer of the syringe thereby mixing the first fluid and the second fluid in the microfluidic mixer to generate the particle-based formulation.

19. The method of claim 18, wherein the displacing of the piston in the proximal direction expels the particle-based formulation out of the microfluidic mixer and into a third chamber of the barrel that is located distal to the piston.

20. The method of claim 18, further comprising:
displacing the piston in the distal direction a second time to expel the particle-based formulation out of an outlet of the barrel.

21. (canceled)

22. The method of claim 18, further comprising:
removing, subsequent to displacing the piston in the proximal direction, a tip cap from an outlet of the barrel.

23. The method of claim 18, further comprising:
prior to the displacing the piston in the proximal direction, removing the first fluid source from the first inlet and the second fluid source from the second inlet; and
connecting a first plug to the first inlet and a second plug to the second inlet.

24. The method of claim 18, wherein the first fluid comprises lyophilized mRNA and the second fluid comprises a lyophilized lipid.
